(19) United States
(12) Patent Application Publication
(10) Pub. No.: US 2006/0281938 A1
(43) Pub. Date: Dec. 14, 2006

(54) HETEROGENEOUS ALKYNE METATHESIS

(76) Inventors: Haim Weissman, Rehovot (IL); Kyle N. Plunkett, New York, NY (US); Hyeon Mo Cho, Urbana, IL (US); Jeffrey S. Moore, Savoy, IL (US)

Correspondence Address: EVAN LAW GROUP LLC, 600 WEST JACKSON BLVD., SUITE 625, CHICAGO, IL, 60661, US

(21) Appl. No.: 11/422,004

(22) Filed: Jun. 2, 2006

Related U.S. Application Data
(60) Provisional application No. 60/688,936, filed on Jun. 9, 2005.

(51) Int. Cl. C07F 11/00; C07C 2/52

(52) U.S. Cl. 556/58; 585/418

(57) ABSTRACT

The present invention provides heterogeneous organometallic catalysts for alkyne metathesis, including the metathesis of internal alkynes. Organometallic precursors are covalently bonded to the oxygen atoms of metal oxide supports to form catalysts having carbyne functionality. The heterogeneous catalysts provide improved turn-over frequencies at lower reaction temperatures than conventional catalysts.

### REFERENCE To RELATED APPLICATIONS

This application claims the benefit of U.S. Provisional Application No. 60/688,936 entitled “Heterogeneous Alkyne Metathesis” filed Jun. 9, 2005, which is incorporated by reference in its entirety.

### FEDERALLY SPONSORED RESEARCH OR DEVELOPMENT

The subject matter of this application may have been funded in part under the following research grants and contracts: Contract Number CHE03-45254 awarded by the National Science Foundation (NSF); and Contract Number DEFG02-91 ER45439 awarded by the U.S. Department of Energy (DOE). The U.S. Government may have rights in this invention.

### BACKGROUND

Metathesis is a chemical reaction where two molecules exchange atoms or groups of atoms. One example of alkyne metathesis, homodimerization, is depicted below in Scheme I.

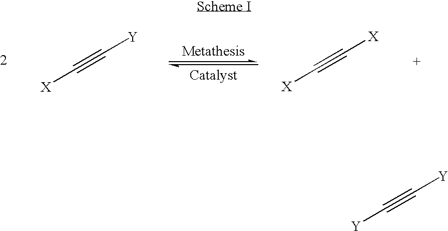

The metathesis catalyst catalyzes the cleavage of two carbon-carbon triple bonds (C≡C) to provide four fragments that rejoin with a new partner. Thus, two starting alkynes (X—≡—Y) each having an X and a Y group undergo metathesis to provide a first product alkyne having two X's (X—≡—X) and a second product alkyne having two Y's (Y—≡—Y). Because metathesis is an equilibrium reaction, removal of one of the alkynes, X—≡—X for example, as the reaction proceeds, such as through precipitation or vaporization, may provide the other alkyne, Y—≡—Y for example, as the major product of the reaction.

While homodimerization is shown in Scheme I, the group exchange ability of the metathesis reaction also can be used for other reaction types, including ring closure, and ring-opening type polymerizations. For example, if the X groups for the two starting alkynes are linked by a continuous chain, metathesis may result in a closed ring structure and a YY alkyne by-product. An example of ring closure is depicted below in Scheme II.

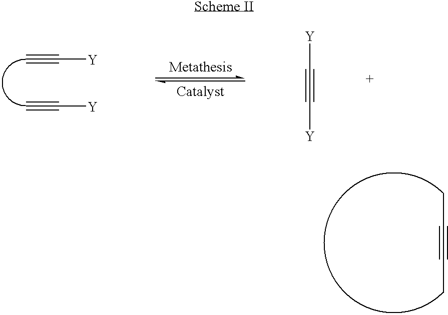

While multiple organometallic catalysts that metathesize carbon-carbon double bonds (C═C), known as olefins, are known, catalysts that metathesize carbon-carbon triple bonds (C≡C), known as alkynes, are uncommon. While a plethora of reasons exist for a lack of suitable alkyne metathesis catalysts, two significant issues are undesirable dimerization and polymerization. Dimerization occurs when two catalyst molecules join with each other, thus arresting catalytic activity and the metathesis reaction. Polymerization is a problem in that instead of metathesizing the alkynes, the catalyst polymerizes the alkynes into long polymer chains, thus loosing alkyne functionality.^{2,3} The lack of suitable alkyne metathesis catalysts increases the cost and complexity of making fine chemicals and natural products for pharmaceuticals, semiconducting polymers for electronic devices, petrochemicals, and the like.

Defined-surface organometallic chemistry has proved advantageous for the production of various catalysts.¹¹⁻¹⁴ When organometallic precursors are used to form ill-defined-surface catalysts, 2% or less of the deposited transition metal is catalytically active. In contrast, when defined-surface organometallic catalysts are formed by covalently bonding controlled amounts of a catalytic organometallic complex to a support, a greater portion of the transition metal is catalytically active. In this manner, defined-surface preparation methods may provide catalysts where parts of the coordination sphere existing about the transition metal center of the organometallic complex in solution is present when the complex is bonded to a support. Multiple molybdenum, tungsten, and rhenium alkylidyne complexes have been bonded to metal oxide supports to provide defined-surface heterogeneous catalysts for olefin metathesis.¹⁵⁻¹⁸

Conventional organometallic catalysts for alkyne metathesis have multiple disadvantages in relation to olefin metathesis catalysts.¹ While homogeneous alkyne metathesis catalysts with phenol-type ligand systems are known to provide catalytic activity at ambient temperature²⁻⁶, the ligand systems are toxic. Furthermore, these homogenous catalysts require complex and expensive chromatography to separate the desired products from the catalyst solution.

Conventional undefined heterogeneous catalysts for alkyne metathesis have been made by impregnating molybdenum oxides on silica, alumina, or titania, but were inefficient, requiring temperatures of greater than 160° C. to obtain turn-over frequencies (TOF) of only several turnover per hour.⁷⁻¹⁰ Relatively poor results also were obtained for a silica supported rhenium catalyst [(SiO)(Re(C—^{t}Bu)(═CH—^{t}Bu)(CH₂Bu)], which could only convert 15 equivalents of 2-pentyne to 2-butyne and 3-hexyme in 20 minutes at approximately 6.7% loading, thus having a t_{1/2} of less than 10 min.¹⁶ Unless stated otherwise, all percents are given on a mol/mol basis.

While not described in the reference, one disadvantage of this rhenium catalyst for alkyne metathesis may be attributable to the methylene hydrogens present on the t-butyl methylene ligands. In one aspect, these hydrogens may transfer from the ligands to the transition metal center and undergo α-elimination with the carbyne to form a transition metal carbene. While transition metal carbenes metathesize olefins, they are ineffective for alkyne metathesis. In another aspect, these hydrogens may undergo reductive elimination from the transition metal center to the oxygen of the silica support, thus removing the organometallic rhenium complex from the support.

As can be seen from the above description, there is an ongoing need for heterogeneous organometallic catalysts that efficiently catalyze alkyne metathesis. The heterogeneous organometallic catalysts of present invention overcome at least one of the disadvantages associated with conventional catalysts.

### SUMMARY

In one aspect, the invention provides a heterogeneous alkyne metathesis catalyst prepared by a process including covalently bonding a precursor to a support.

In another aspect, the invention provides a method of metathesizing an alkyne by reacting the heterogeneous alkyne metathesis catalyst with the alkyne at a temperature between 15 and 100° C. to metathesize the alkyne.

In another aspect, the invention provides a heterogeneous alkyne metathesis catalyst, including a means for supporting a precursor, the precursor comprising means for metathesizing the alkyne.

In another aspect, the invention provides a compound having structure formula (III).

In another aspect, the invention provides a method of metathesizing an alkyne by reacting the compound having the structure (III) with the alkyne at a temperature between 15 and 100° C. to metathesize the alkyne.

In another aspect, the invention provides an improved alkyne metathesis catalyst, the improvement including covalently bonding the transition metal center of the catalyst to at least one oxygen atom of a support.

The following definitions are included to provide a clear and consistent understanding of the specification and claims.

The term “organometallic complex” is defined as a complex where a transition metal is bonded to at least one carbon atom through a sigma bond (formal charge of −1 on the carbon atom sigma bonded to the transition metal) or a pi bond (formal charge of 0 on the carbon atoms pi bonded to the transition metal). For example, ferrocene is an organometallic complex with two cyclopentadienyl (Cp) rings, each bonded through its five carbon atoms to an iron center by two pi bonds and one sigma bond. Another example of an organometallic complex is ferricyanide (III) and its reduced ferrocyanide (II) counterpart, where six cyano ligands (formal charge of −1 on each of the 6 ligands) are sigma bonded to an iron center through the carbon atoms.

A “homogenous” alkyne catalyst solubilizes in the same solution in which the alkyne is solubilized.

A “heterogeneous” alkyne catalyst is insoluble in the solution or gas phase in which the alkyne is present.

A “heterogeneous alkyne metathesis catalyst” is a heterogeneous catalyst that metathesizes alkynes at a TOF of at least 0.007 molₚ*mol_{c}⁻¹s⁻¹ between 20 and 26° C. with a catalyst loading of 0.3%.

The terms “soluble” or “solubilized” mean a solid, liquid, or gas solvated in a liquid to provide a solution, where a solution, unlike a dispersion, suspension, or mixture, lacks an identifiable interface between the solubilized species and the solvent. Thus, in homogenous catalyst solutions, the organometallic complex is in direct contact with the solvent. Conversely, for heterogeneous catalysts, an interface exists between the solvent and the catalyst. For a catalyst to be “soluble in the solution,” at least one ppm of the catalyst solubilizes in the solution.

The term “catalyst loading” is defined as the mol % of catalyst relative to the alkyne. For example, 1 mol. of catalyst relative to 100 mol. of alkyne is 1% catalyst loading.

The term “turn-over frequencies” (TOF) is defined as the number of alkyne molecules converted into a product by one molecule catalyst as a function of time. For example, if 100 moles of an alkyne were converted to a product by 1 mol. of a catalyst in 10 seconds, the TOF for the catalyst is 10 molₚ*mol_{c}⁻¹s⁻¹.

The t_{1/2} value is the time it takes for a catalyst to convert ½ of an alkyne starting material into a product. In a related aspect, the t_{1/2} value for a catalyst may be the time is takes to convert ½ of a convertible alkyne starting material into a product. For example, in an equilibrium reaction where only 80% of the alkyne starting material may be converted to a product, the t_{1/2} value is reached when 40% of the alkyne has been converted to the product.

The term “substituted” refers to a group that is bonded to a parent molecule or group. Thus, a benzene ring having a methyl substituent is a methyl-substituted benzene. Similarly, a benzene ring having 5 hydrogen substituents would be an unsubstituted phenyl group when bonded to a parent molecule.

The term “covalent bond” represents a type of homopolar bonding where an electron is shared between two atoms to form the bond. Covalent bonds have definite directions in space, thus allowing for the spatial relationship between atoms to be maintained. Conversely, ionic bonds are formed by the transfer of an electron from one atom to another to create an attractive charge that forms the bond. Ionic bonds lack definite directions in space, thus preventing distinct spatial relationships between atoms. Unlike covalently bound compounds, ionic compounds dissociate in water and are commonly referred to as salts. Hydrogen bonds differ from covalent bonds because hydrogens bonds require three atoms and include heteroatoms or halogens. Covalent bonding is unlike hydrogen bonding where electrons are not shared between the atoms.

The term “aliphatic” refers to a monovalent group including carbon and hydrogen that is not aromatic. In addition to carbon and hydrogen, aliphatic groups may include heteroatoms. For example, aliphatic groups may include alkyl, cycloalkyl, alkoxy, hydroxy, halo, and amino groups.

The term “alkyl” refers to an unsubstituted or substituted monovalent saturated hydrocarbon group which may be linear or branched. Unless otherwise defined, such alkyl groups typically contain from 1 to 10 carbon atoms. Representative alkyl groups include, by way of example, methyl, ethyl, n-propyl, isopropyl, n-butyl, sec-butyl, isobutyl, tert-butyl, n-pentyl, n-hexyl, n-heptyl, n-octyl, n-nonyl, n-decyl, and the like.

The term “cycloalkyl” refers to a monovalent saturated carbocyclic hydrocarbon group having a single ring or fused rings. Unless otherwise defined, such cycloalkyl groups contain from 3 to 10 carbon atoms. Representative cycloalkyl groups include, by way of example, cyclopropyl, cyclobutyl, cyclopentyl, cyclohexyl, and the like.

The term “aromatic” refers to a monovalent group including carbon and hydrogen exhibiting aromatic character. For example, aryl and heteroaryl groups are aromatic.

The term “aryl” refers to an unsubstituted or substituted monovalent aromatic hydrocarbon having a single ring (e.g., phenyl) or fused rings (e.g., naphthalene). Unless otherwise defined, such aryl groups typically contain from 6 to 10 carbon ring atoms. Representative aryl groups include, by way of example, phenyl and naphthalene-1-yl, naphthalene-2-yl, and the like.

The term “heteroaryl” refers to a substituted or unsubstituted monovalent aromatic group having a single ring or fused rings and containing in the ring at least one heteroatom (typically 1 to 3 heteroatoms) selected from nitrogen, oxygen, or sulfur. Unless otherwise defined, such heteroaryl groups typically contain from 5 to 10 total ring atoms. Representative heteroaryl groups include, by way of example, monovalent species of pyrrole, imidazole, thiazole, oxazole, furan, thiophene, triazole, pyrazole, isoxazole, isothiazole, pyridine, pyrazine, pyridazine, pyrimidine, triazine, indole, benzofuran, benzothiophene, benzimidazole, benzthiazole, quinoline, isoquinoline, quinazoline, quinoxaline and the like, where the point of attachment is at any available carbon or nitrogen ring atom.

The term “hydroxy” or “hydroxyl” refers to an —OH group.

The term “alkoxy” refers to an —OR group, where R can be a substituted or unsubstituted alkyl, alkylene, cycloalkyl, or cycloalkylene. Representative alkoxy groups include, by way of example, methoxy, ethoxy, isopropyloxy, and trifluoromethoxy.

The term “halo” or “halogen” refers to fluoro-(—F), chloro-(—Cl), bromo-(—Br), and iodo-(—I).

The term “metal” refers to the metal and metalloid elements from Groups I, II, III, IV, V, and VI of the Periodic Table. Hydrogen, carbon, nitrogen, phosphorous, oxygen, sulfur, and selenium are excluded.

The term “hydride functionality” refers to a hydrogen that may be transferred to a chemical moiety as H—. For example, a metal hydrides include (n-Bu)₃SnH and NaBH₄.

The term “condensation” refers to a reaction in which two or more molecules are covalently joined. Likewise, condensation products are the products formed by the condensation reaction.

### BRIEF DESCRIPTION OF THE DRAWINGS

The invention can be better understood with reference to the following drawings and description. The figures are not intended to accurately represent molecules or their interactions, emphasis instead being placed upon illustrating the principles of the invention.

FIG. 1 is a plot of the percent homodimerization of an aliphatic alkyne verses time.

FIG. 2 is a plot of the amount of metathesis product formed in relation to catalyst loading verses time.

FIG. 3A is an X-ray photoelectron spectrum of a catalytic precursor before bonding with a support.

FIG. 3B is an X-ray photoelectron spectrum of a heterogeneous alkyne metathesis catalyst in accord with the present invention.

FIG. 4 is a ¹³C magic angle spinning nuclear magnetic resonance spectrum of a heterogeneous alkyne metathesis catalyst after covalent bonding to a support in accord with the present invention.

FIG. 5 depicts before and after infrared spectra of a support and a heterogeneous alkyne metathesis catalyst in accord with the present invention.

FIG. 6 depicts multiple alkynes homodimerized by the heterogeneous alkyne metathesis catalyst depicted as Structure (II).

FIG. 7 depicts multiple substrates suitable for ring closing alkyne metathesis (RCAM), the ring closure products corresponding to each substrate, and the percent yield when reacted with the heterogeneous alkyne metathesis catalyst depicted as Structure (II).

FIG. 8 represents a reaction similar to ring closure, except that the alkynes joined by the heterogeneous alkyne metathesis catalyst depicted as Structure (II) are held on separate molecules to form macrocycles.

### DETAILED DESCRIPTION

The present invention provides heterogeneous organometallic catalysts for alkyne metathesis, including the metathesis of internal alkynes. Organometallic precursors are covalently bonded to the oxygen atoms of metal oxide supports to form catalysts having carbyne functionality. Surprisingly, the heterogeneous catalysts provide improved turn-over frequencies at lower reaction temperatures than conventional catalysts. The use of ligands having amide functionality in the precursor complex may be responsible for these unexpected benefits.

Scheme III, below, represents the covalent attachment of a carbyne precursor 100 to a metal oxide support 200 to provide a heterogeneous alkyne metathesis catalyst 300.

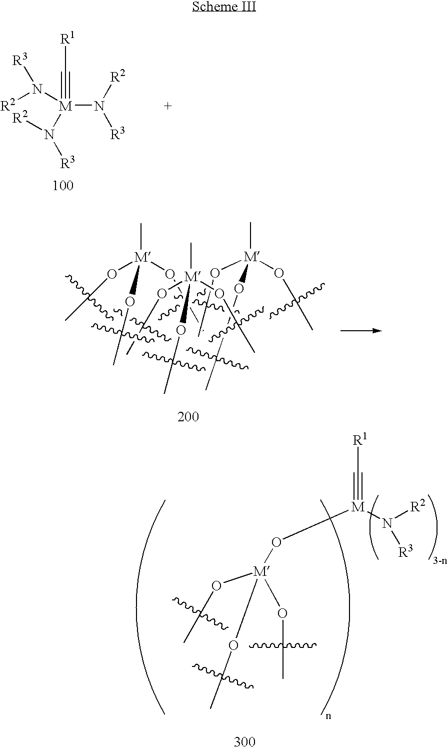

While higher temperatures may be possible, the catalyst 300 may catalyze alkyne metathesis at temperatures from 15 to 100° C., from 15 to 50° C., or preferably from 20 to 26° C. The ability of the catalyst 300 to catalyze alkyne metathesis at or near room temperature is an unexpected and surprising benefit.

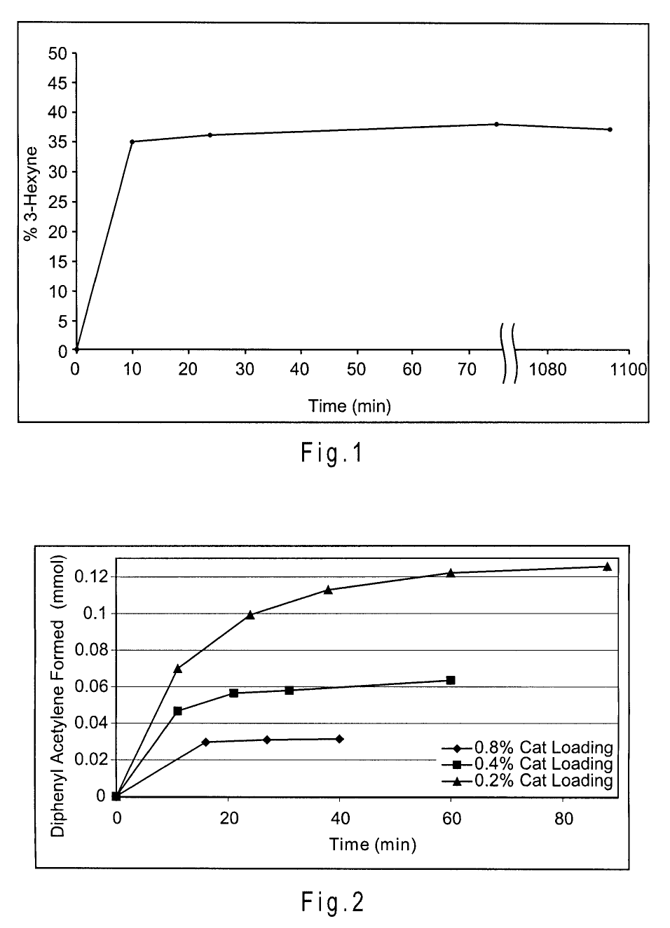

FIG. 1 is a plot of the percent homodimerization of an aliphatic alkyne verses time. The catalyst 300 was prepared from the precursor 100 having Structure (II), below, with 0.8% catalyst loading and was allowed to catalyze the metathesis of 6.1 mg of a 1-phenyl-1-butyne in 600 μL of m-xylene solvent at room temperature. The t_{1/2} for the reaction was less than 5 minutes, thus establishing at least an order of magnitude improvement over a conventional catalyst having t-butyl methylene ligands.

In one aspect, with an alkyne concentration of 0.28 moles/Liter, 0.3% catalyst loading, and a reaction temperature of from 20 to 26° C., the catalyst 300 has a TOF of at least 0.007, at least 0.02, at least 0.1, or at least 0.2 molₚ*mol_{c}⁻¹s⁻¹. In another aspect, with the same alkyne concentration and reaction conditions, the t_{1/2} for the catalyst 300 is less than 20, 10, 8, 5, or 3 minutes.

While not wishing to be bound by any particular theory, the unexpected ability of the heterogeneous catalyst 300 to metathesize alkynes with good turnover at room temperature is believed to be attributable to the amide ligands present on the precursor 100. In relation to conventional catalysts, the amide ligands lack hydrogen atoms a to the transition metal center (M-Z-H), thus preventing possible deactivation of the catalyst with regard to alkyne metathesis from conversion of the transition metal carbyne to a transition metal carbene. Furthermore, the nitrogen ligands, which have a lone electron pair and a substantially different electronegativity than carbon, provide a different electronic environment at the active transition metal center than carbon ligands, when not completely replaced by the oxide ligands of the support 200.

FIG. 2 is a plot of the amount of homodimerization metathesis product formed (mmol) for catalyst loadings of 0.2, 0.4, and 0.8% as a function of time. While higher loadings are possible, the catalyst 300 may catalyze alkyne metathesis at loadings below 1, 0.8, 0.5, 0.1, or 0.08 mol %. In one aspect, loadings from 0.2 to 1 or from 0.5 to 0.9% are preferred.

In another aspect, the catalyst 300 demonstrates catalytic activity for at least three cycles. Unexpectedly, the catalyst 300 resists undesirable alkyne polymerization and may homodimerize 3-propynyl thiophene, an alkyne unable to undergo metathesis with conventional homogeneous or heterogeneous catalysts.¹⁹

The catalyst 300 may catalyze metathesis of alkynes in solution or in the gas phase. When used with alkynes in solution, the catalytic reaction may be driven to produce a selected product by removal of a competing by-product.^{2,3,4,20-25} For example, the by-product may be removed from the solution in a gaseous state by vacuum or gas purging. If the by-product is insoluble in the solution, it may be removed by precipitation to drive the reaction to the desired product.

The catalyst 300 is formed by covalent bonding of the precursor 100 to the support 200. During bonding, the amide ligands are believed to convert to secondary amines through condensation with hydrogen atoms removed from the hydroxyl ligands present on the support 200. The bonding process results in from one to three of the transition metal-nitrogen bonds provided by the amide ligands of the precursor 100 being replaced by transition metal-oxygen bonds provided by oxide ligands from the support 200. Thus, for Scheme III, above, n is an integer from 1 to 3, preferably from 1 to 2. In this manner, a substantially pure heterogeneous alkyne metathesis catalyst may be formed.

While metal-oxygen bonding has replaced transition metal-nitrogen bonding, the covalent nature of the bonding remains. Thus, the coordination sphere about the transition metal center of the precursor 100 is substantially maintained for the heterogeneous catalyst 300. This bonding reaction (ligand substitution) is illustrated in Scheme III, above, which shows that the catalyst 300 includes 3-n amide ligands for n oxide bonds with the support 200.

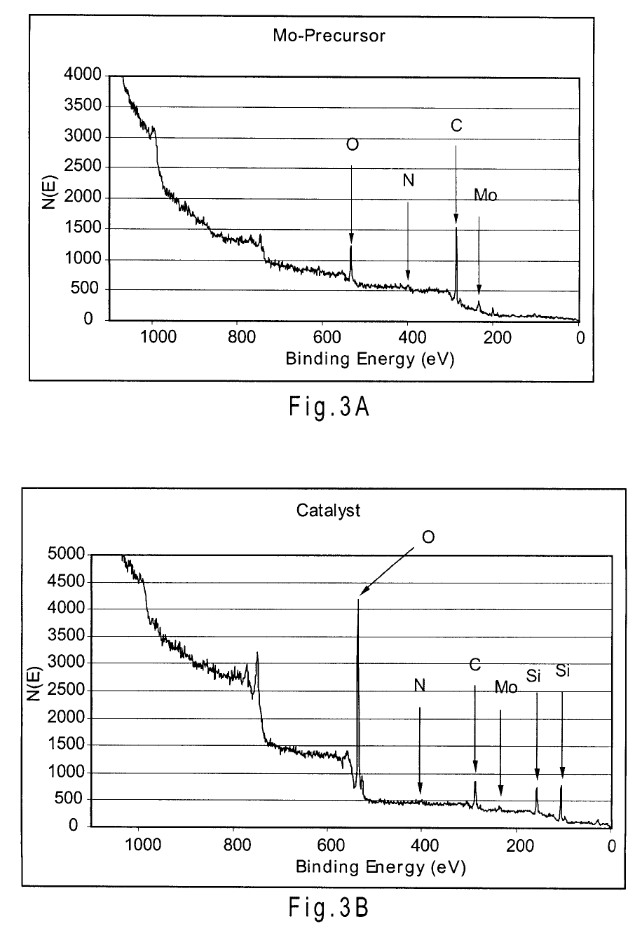

The covalent bonding of the catalyst 300 was established by multiple spectroscopic techniques. FIG. 3A is an X-ray photoelectron spectra (XPS) showing the oxygen (O), nitrogen (N), carbon (C), and molybdenum (Mo) peaks of a precursor having Structure (II), as described further below. FIG. 3B is a XPS of the precursor of FIG. 3A covalently bonded to a silica support to form a heterogeneous alkyne catalyst. As seen in FIG. 3B from the shifting of the affinity energy to a higher region, the transition metal-oxygen bonds are significantly more electronegative than the transition metal-nitrogen bonds they replace.

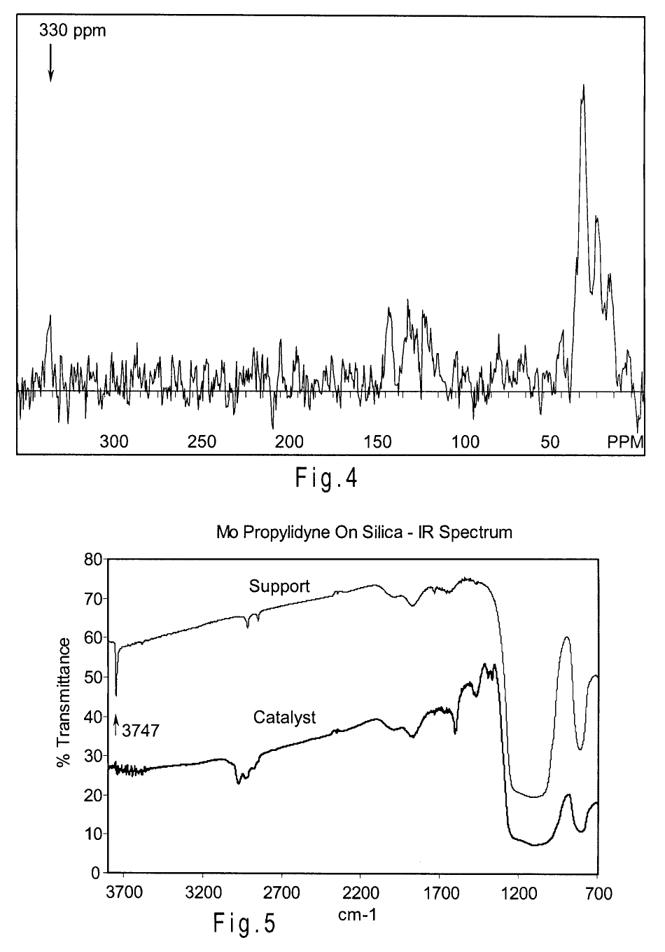

FIG. 4 shows a magic angle spinning nuclear magnetic resonance (MAS-NMR) carbon thirteen spectra establishing the existence of a carbyne bond between molybdenum and carbon. The peak at ˜330 ppm represents the carbyne carbon bonded to the molybdenum.

FIG. 5 depicts before and after infrared (IR) spectra establishing that —OH groups present on the support 200 at ˜3747 cm⁻¹ disappear when the precursor 100 is bonded to the support 200 to form the catalyst 300. The reduction in the hydroxyl peak may be attributed to the elimination of aniline as the oxide bonds replace nitrogen bonds at the transition metal center.

The precursor 100 preferably bonds to the support 200 at a concentration of from 0.5 to 100, preferably from 50 to 80, and more preferably from 60 to 70% (mol. metal/mol. hydroxyl). Before bonding the precursor 100 with the support 200, the support 200 may be treated to enhance bonding with the precursor 100. In one aspect, treating includes heating the support 200 from 200 to 700° C., preferably from 180 to 220° C., under vacuum. In another aspect, treating includes heating the support 200 from 300 to 700° C., preferably from 380 to 420° C., under an oxygen atmosphere.

The carbyne precursor 100 has the general Structure formula (I):

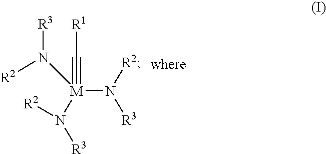

M is a transition metal selected from the group consisting of Mo and W.

In one aspect, R¹ is any carboyl. In another aspect, R¹ is selected from the group consisting of aliphatic and aromatic moieties. In another aspect, R¹ is selected from the group consisting of aliphatic and aromatic moieties excluding hydroxy, primary amine, secondary amine, thiol, sulfoxide, sulfate, phosphine, phosphite, phosphonate, primary silane, secondary silane, tertiary silane, and protic acid functionality. In another aspect, R¹ is selected from the group consisting of aliphatic and aromatic moieties, which do not contain hydrogen atoms bonded to elements other than carbon.

In another aspect, R¹ is selected from the group consisting of aliphatic and aromatic moieties, which do not include hydride functionality at the alpha position when R¹ includes one carbon atom, at the alpha and beta positions when R¹ includes at least two carbon atoms, and at the alpha, beta, and gamma positions when R¹ includes at least three carbon atoms. In another aspect, when R¹ includes at least four carbon atoms, R¹ is selected from the group consisting of aliphatic and aromatic moieties excluding hydroxy, primary amine, thiol, sulfoxide, phosphite, and phosphine functionality at delta and higher positions.

In one aspect R² is a secondary or tertiary carboyl. In another aspect, R² is independently selected from the group consisting of aliphatic and aromatic moieties. In another aspect, R² is independently selected from the group consisting of aliphatic and aromatic moieties excluding hydroxy, primary amine, secondary amine, thiol, sulfoxide, sulfate, phosphine, phosphite, phosphonate, primary silane, secondary silane, tertiary silane, and protic acid functionality. In another aspect, R² is independently selected from the group consisting of aliphatic and aromatic moieties, which do not contain hydrogen atoms bonded to elements other than carbon.

In another aspect, R² is independently selected from the group consisting of aliphatic and aromatic moieties, which do not contain hydride functionality at the alpha position when R² includes one carbon atom and at the alpha and beta positions when R² includes at least two carbon atoms. In another aspect, when R² includes at least three carbon atoms, R² is independently selected from the group consisting of aliphatic and aromatic moieties excluding hydroxy, primary amine, thiol, sulfoxide, phosphite, and phosphine functionality at gamma and higher positions.

R³ is a phenyl group. In another aspect, R³ is a phenyl group having m substituents where m is an integer from 1 to 5. In another aspect, each substituent may be independently selected from the group consisting of hydrogen, aliphatic moieties, and aromatic moieties. In another aspect, each substituent may be independently selected from the group consisting of aliphatic and aromatic moieties excluding hydroxy, primary amine, secondary amine, thiol, sulfoxide, sulfate, phosphine, phosphite, phosphonate, primary silane, secondary silane, tertiary silane, and protic acid functionality. In another aspect, each substituent may be independently selected from the group consisting of aliphatic and aromatic moieties that do not contain hydrogen atoms bonded to elements other than carbon.

In one aspect, preferred precursors have the general structure formula (I), where M is Mo. In another aspect, preferred precursors have the general structure formula (I), where R¹ and R² are independently selected aliphatic groups. In another aspect, preferred precursors have the general structure (I), where R¹ and R² are independently selected aromatic groups. In another aspect, these aromatic groups may be substituted or unsubstituted. In another aspect, the aromatic groups are aryl or heteroaryl.

When R¹ and R² are aliphatic groups including only carbon and hydrogen, each may be independently selected alkyl or cycloalkyl groups, such as t-butyl or ethyl. In a preferred aspect, R¹ is ethyl, thus having two hydrogen substituents at the alpha carbon and three hydrogen substituents at the beta carbon, and R² is t-butyl, thus having three methyl substituents at the alpha carbon and three hydrogen substituents at the beta carbon.

In one aspect, preferred precursors have the general structure formula (I), where R³ is a phenyl group having 3, 2, or 1 substituent. In a preferred aspect where m is 2, the substituents are independently selected aliphatic groups. When the substituents are unsubstituted aliphatic groups, each substituent may be an independently selected alkyl or cycloalkyl group. In one preferred aspect, the phenyl rings are each substituted with a methyl group at each of the two meta positions. In another preferred aspect where m is 2, the substituents are independently selected aromatic groups. In another aspect, the aromatic groups are aryl or heteroaryl. To tune the reactivity of the catalyst, the substituents of the phenyl rings may be electron withdrawing groups, such as halogens, or electron donating groups, such as alkoxy groups.

In a preferred aspect, the precursor 100 is structure formula (II):

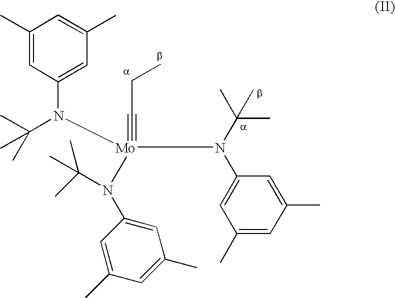

The support 200 may be any metal oxide including hydroxyl surface ligands that is compatible with the precursor 100 and the desired metathesis reaction. Preferably, the metal oxide that forms the support 200 is selected from the group consisting of oxides of silica, alumina, magnesia, titania, or combinations thereof. Thus, M′, as shown in the support 200 of Scheme III above, may be Si, Al, Mg, Ti, or combinations thereof. More preferably, the support 200 includes oxides of silica, alumina, titania, or combinations thereof. At present, non-toxic supports, such as amorphous silica, are especially preferred.

The metal oxide or oxides that form the support 200 may be crystalline or amorphous with any pore size compatible with the desired metathesis reaction. In one aspect, the support may have a surface area from 30 to 1100 or from 60 to 600 m²/g, preferably from 100 to 300 m²/g.

The surface of the metal oxide forming the support 200 is preferably protic, having a hydroxyl ligand concentration of from 1 to 5 equivalents of —OH per square nanometer of surface area. More preferably, the support 200 provides from 2 to 3 equivalents of —OH per square nanometer of surface area. In another aspect, metal oxides having pKa's from 4 to 10 are preferred, with metal oxides having pKa's from 7 to 9 being more preferred.

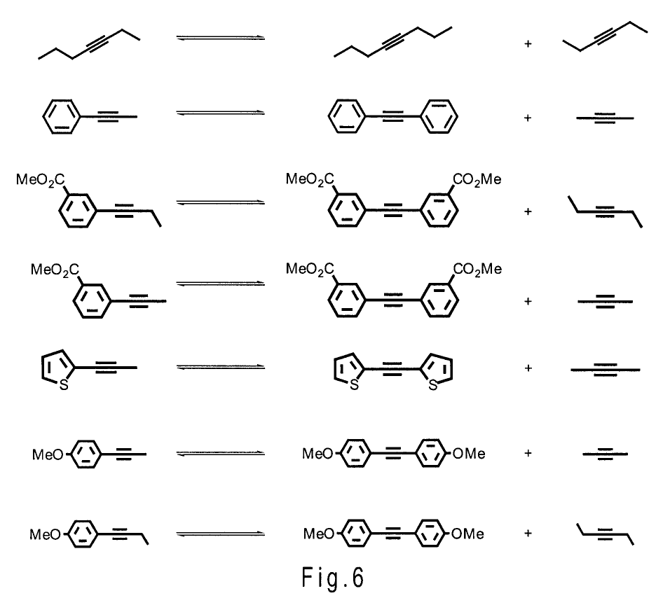

FIG. 6 depicts multiple alkynes homodimerized by the heterogeneous alkyne metathesis catalyst depicted as Structure (II). For each alkyne, the X and Y groups of the original alkyne are exchanged to give two alkynes, the first including two X groups and the second including two Y groups, in accordance with Scheme I as previously discussed.

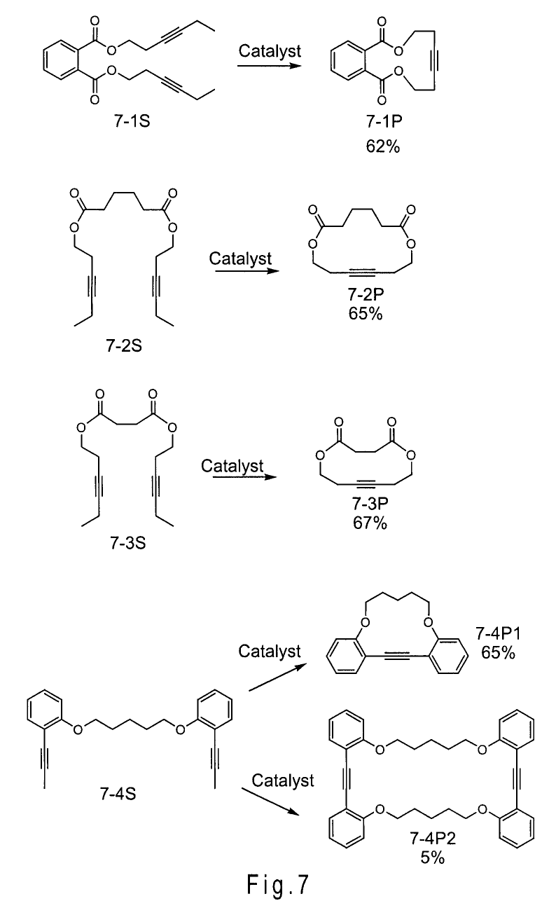

FIG. 7 depicts multiple substrates suitable for ring closing alkyne metathesis (RCAM), the ring closure products corresponding to each substrate, and the percent yield when reacted with the heterogeneous alkyne metathesis catalyst depicted as Structure (II). Due to the geometric restrictions imposed on the two alkyne functionalities of each RCAM diyne substrate, the metathesis reaction results in ring closure, in accordance with Scheme II, as previously discussed. While the figure shows four examples of diynes useful as RCAM substrates, any diyne that undergoes homogeneous ring closure may be used. In one aspect, alkynes having silanes or saturated tertiary carbon atoms bonded directly to one of the alkyne carbons may be less preferred for RCAM reactions. This reduced preference may be attributable to unfavorable steric interactions. A listing of useful substrates for RCAM reactions may be found in the work of Furstner and Bunz, for example.²⁶⁻²⁹

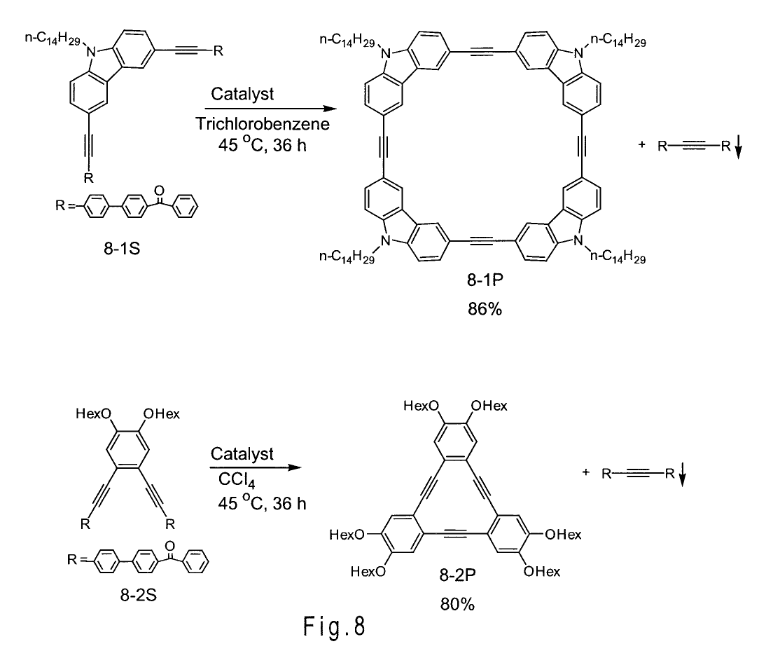

FIG. 8 represents a reaction similar to ring closure, except the alkynes that join are held on separate molecules. Substrates 8-1S and 8-2S were joined by the heterogeneous alkyne metathesis catalyst depicted as Structure (II) to form the tetrameric macrocycle 8-1P and the trimeric macrocycle 8-2P, respectively, in over 80% yield. Thus, the catalyst cyclooligomerized at least three molecules to form the macrocycles. A listing of useful substrates for cyclooligomerizetion reactions may be found in the work of Zhang, for example.³⁰⁻³²

The following examples are provided to illustrate one or more preferred embodiments of the invention. Numerous variations can be made to the following examples that lie within the scope of the invention.

### EXAMPLES

### Example 1

### Catalyst Preparation

Amorphous silica (particle size 30-40 nm, 200 m²/g, ˜4 μmol/m²) was treated at 400° C. under an O₂ atmosphere for 14 h. Elemental analysis of the resulting silica showed CHN values of 0.03, 0, and 0%, respectively. Inside an argon-filled glove box, silica (0.350 g, ˜280 μmol) was dispersed in 10 mL toluene. The tris-amido Mo carbyne complex (50.0 mg, 75.1 μmol) was dissolved in 2.5 mL toluene and added dropwise to the stirring dispersion of silica. The mixture was stirred for 10 h, concentrated to ˜4 mL, and stirred for an additional 48 h. The resulting solution was transferred to 1.5 mL test tubes and centrifuged. The resulting silica was rinsed with toluene, THF, 2× acetonitrile, and benzene. The resulting silica was suspended in benzene and lyophilized. The resulting silica was submitted for elemental analysis. Elemental analysis for molybdenum gave the value of 1.60% (w/w), establishing that 1.6% (w/w) of the catalyst is molybdenum metal. Presuming that all molybdenum is active, catalyst loading may be determined from the percent molybdenum obtained from elemental analysis and the weight of the catalyst.

### Example 2

### Homodimerization Metathesis

In accordance with Example 1, catalyst was prepared with 1.5-1.6% Mo/silica (w/w). The catalyst (3.0 mg, 0.50 μmol Mo) was weighed into a 1.5 mL reaction vessel. Approximately 0.25 mmol of alkyne, such as 3-heptyne or a mixture of 3-hexyneand 3-octyne, was transferred to the reaction vessel with approximately 300 μL of toluene or m-xylene. The reaction vessel was sealed and the reaction mixture was agitated at room temperature during the reaction. Thus, metathesis was catalyzed in alkynes having significant substituent diversity.

### Example 3

### In situ Catalyst Preparation and Homodimerization

Amorphous silica (particle size 30-40 nm, 200 m²/g, ˜4 μmol/m²) was treated at 400° C. under an O₂ atmosphere for 14 h. Elemental analysis of the resulting silica showed CHN values of 0.03, 0, and 0%, respectively. Inside an argon-filled glove box, silica (7.0 mg, ˜5.6 μmol) was added into a screw cap NMR tube. 600 μL of a yellow/brown d₈-toluene solution of tris-amido Mo carbyne complex (1.67M, 1.5 μmol) was added to the NMR tube. The suspension was shaken at 25° C. for 30 min. The solution turned colorless as the silica turned brown. 1-phenyl-1-butyne (21.2 μL, 0.15 mmol) was added to the NMR tube and the suspension was shaken at 21° C. and followed up by ¹HNMR. The reaction reached equilibrium after 22 min at 44% yield of diphenylacetylen and 3-hexyne with a t_{1/2} of less than 2.3 min.

### Example 4

### In Situ Catalyst Preparation and Ring Closing Metathesis

A mixture of 5 mol % of the tris-amido Mo carbyne complex of Example 1 and fumed silica (7× weight of Mo complex) was stirred for 5 min in 1,2,4-trichlorobenzene. A solution of the RCAM diyne substrate was added to the suspension and the pressure above the mixture was reduced to about 1 mm Hg to remove 3-hexyne. Depending on the diyne substrate, the equilibrium also may be driven to completion by precipitation. Corresponding cyclic alkynes were generally produced in over 60% yield when isolated by filtration and silica gel chromatography purification. Molybdenum metal was not observed with ICP elemental analysis in the cyclic alkyne products. As shown for substrate 4 in FIG. 7, dimeric macrocycles also are possible from RCAM substrates.

### Example 5

### In Situ Catalyst Preparation and Cyclooligomerization

As represented in FIG. 8, when the RCAM substrates of Example 4 were replaced with arylene ethynylenes, cyclooligomerization was observed to form macrocycles. Due to the larger product size, precipitation was generally used to remove bis(benzoylbiphenyl)ethylene as the poorly soluble by-product. The cyclooligomerization products were obtained in high purity by filtering off the by-product. Single crystal X-Ray analysis of the trimeric product 8-2P showed a triangular planar core.

### Example 6

### Catalyst Loading Comparisons for Homodimerization

For 0.2 mol % catalyst loading, 0.25 mmol of alkyne was added to a reaction vial containing 3.0 mg of the catalyst from Example 1 (0.50 μmol Mo) and 600 μL of toluene and agitated by shaking or stirring. For 0.4 and 0.8 mol % catalyst loading, the alkyne concentration was increased to 0.5 mmol and 1.0 mmol, respectively. For reactions containing 4.0 mol % catalyst loading, 0.065 mmol of the alkyne was added to 15.5 mg of the catalyst (2.52 μmol Mo) and 600 μL of toluene. Thus, while the amount of catalyst is constant, the amount of alkyne is increased to reduce the catalyst loading.

The t_{1/2} values present in Table I, below, represent the time it takes for the reaction to proceed to one-half of the final equilibrium concentrations.

TABLE I

| | | |
|---|---|---|
| | Catalyst loading | t_{1/2} at 24° C. |
| Alkyne | (mol %) | (min) |

| | | |
|---|---|---|
| 1-phenyl-1-butyne | 0.8 | <5 |
| | 0.4 | <8 |
| | 0.2 | 11 |
| 3-butynyl methyl benzoate | 4 | <5 |
| | 0.8 | <10 |
| | 0.4 | 40 |
| 1-phenyl-propyne | 0.2 | 20 |
| 3-propynyl methyl benzoate | 0.2 | 14 |
| 2-propynyl thiophene | 4 | <10 |
| 3-heptyne | 0.8 | <5 |
| 3-hexyne + 3-octyne | 0.8 | <5 |

The data from the table establish that the catalyst has a substantially faster TOF than conventional heterogeneous alkyne metathesis catalysts for a variety of alkynes.

### Example 7

### Metathesis Rate Comparison

For 0.3 mol % catalyst loading, 0.167 mmol of alkyne was added to a reaction vial containing 3.0 mg of the catalyst from Example 1 (0.50 μmol Mo) and 600 mL of toluene and agitated by shaking or stirring. Tables IIA-IIC below show the rate, TOF, and T_{1/2} values from the alkyne homodimerization reactions at 0.3% catalyst loading. While there is variation depending on the specific alkyne, the TOF and T_{1/2} values demonstrate applicability of the catalyst to alkyne metathesis.

TABLE IIA
K_{2 obs} (M⁻¹ · min⁻¹)

| Substitiuent | Butynyl | Propynyl |
|---|---|---|
| MeO— | 0.243 | 0.058 |
| H— | 0.186 | 0.031 |
| m-CO₂Me | 0.068 | 0.023 |

TABLE IIB
TOF (molₚ · mol_{c}⁻¹ · s⁻¹)

| Substitiuent | Butynyl | Propynyl |
|---|---|---|
| MeO— | 0.051 | 0.014 |
| H— | 0.056 | 0.0085 |
| m-CO₂Me | 0.021 | 0.0076 |

TABLE IIC
T_{1/2} (min)

| Substitiuent | Butynyl | Propynyl |
|---|---|---|
| MeO— | 7 | — |
| H— | 6 | — |
| m-CO₂Me | 15.5 | — |

### Example 8

### Catalyst Recycling

Recycling of the catalyst from Example 1 was accomplished with 0.8 mol % and 4 mol % loading. For 0.8 mol % catalyst loading, 1.0 mmol of butynyl methyl benzoate was added to a reaction vial containing 3.0 mg of the catalyst (0.50 μmol) and 600 μL of toluene and agitated for 2 h. The reaction mixture was centrifuged and decanted. The silica was washed with 200 μL of solvent, centrifuged and decanted. A second mixture of 1.0 mmol alkyne in 600 μL of toluene was added to the silica and agitated for 2 h, and repeated.

Each of the cycles were analyzed and found that for cycles 1, 2, and 3 the conversions were 43.8, 32.7, and 8.7%, respectively. For the 4.0 mol % catalyst loading, 64 μmol of propynyl thiophene were added to a reaction vial containing 15.5 mg of the catalyst (2.52 μmol) and 600 μL for toluene and stirred or shaken for 5 h. The reaction mixture was centrifuged and the mixture was decanted. The silica was washed with 200 μL of solvent, centrifuged and decanted. The conversion percentages established that the catalyst may be used at least twice for the metathesis of propynyl thiophene.

A second mixture of 64 μmol 1-phenyl-1-butyne in 600 μL of toluene was added to the silica and stirred or shaken for 5 h, and repeated. Each of the cycles were analyzed and found that for cycles 1, 2, and 3, the conversions were 45.2, 52.1, and 32.3% respectively. The conversion percentages established that the catalyst may be used at least three times for the metathesis of 1-phenyl-1-butyne.

### REFERENCES

(1) Astruc, D. New J. Chem. 2005, 29, 42-56.
(2) Zhang, W.; Kraft, S.; Moore, J. S. Chem. Commun. 2003, 832-833.
(3) Zhang, W.; Kraft, S.; Moore, J. S. J. Am. Chem. Soc. 2004, 126, 329-335.
(4) Zhang, W.; Moore, J. S. J. Am. Chem. Soc. 2004, 126, 12796.
(5) Blackwell, J. M.; Figueroa, J. S.; Stephens, F. H.; Cummins, C. C. Organometallics 2003, 22, 3351-3353.
(6) Zhang, W.; Moore, J. S. Macromolecules 2004, 37, 3973-3975.
(7) Kukes, S. G.; Banks, R. L.: Metathesis Process and Catalyst. U.S. Pat. No. 4,465,890, 1984.
(8) Mortreux, A.; Delgrange, J. C.; Blanchard, M.; Lubochinsky, B. J. Mol. Catal. 1977, 2, 73-82.
(9) Mortreux, A.; Petit, F.; Blanchard, M. J. Mol. Catal. 1980, 8, 97-106.
(10) Schrock, R. R.: Catalyst Composition to Effect Metathesis of Acetylenes. U.S. Pat. No. 4,427,595, 1984.
(11) Coperet, C.; Chabanas, M.; Saint-Arroman, R. P.; Basset, J.-M. Angew. Chem. Int. Edit. 2003, 42, 156-181.
(12) Evans, J. Chem. Soc. Rev. 1997, 26, 11-19.
(13) Lepetit, C.; Che, M. J. Mol. Catal. A 1995, 100, 147-160.
(14) Basset, J.-M.; Lefebvre, F.; Santini, C. Coord. Chem. Rev. 1998, 178-180., 1703-1723.
(15) Saint-Arroman, R. P.; Chabanas, M.; Baudouin, A.; Coperet, C.; Basset, J.-M.; Lesage, A.; Emsley, L. J. Am. Chem. Soc. 2001, 123, 3820-3821.
(16) Chabanas, M.; Baudouin, A.; Coperet, C.; Basset, J.-M. J. Am. Chem. Soc. 2001, 123, 2062-2063.
(17) Chabanas, M.; Baudouin, A.; Coperet, C.; Basset, J.-M.; Lukens, W.; Lesage, A.; Hediger, S.; Emsley, L. J. Am. Chem. Soc. 2003, 125, 492-504.
(18) Chabanas, M.; Baudouin, A.; Coperet, C.; Basset, J.-M. Chem. Eur. J. 2003, 9, 971-975.
(19) Bunz, U. H. F. Acc. Chem. Res. 2001, 34, 998-1010.
(20) Weiss, K.; Michel, A.; Auth, E.-M.; Bunz, U. H. F.; Mangel, T.; Miillen, K. Angewandte Chemie, International Edition 1997, 36, 506-509.
(21) Kloppenburg, L.; Song, D.; Bunz, U. H. F. J. Am. Chem. Soc. 1998, 120, 7973-7974.
(22) Bunz, U. H. F.; Kloppenburg, L. Angew. Chem. Int. Edit. 1999, 38, 478-481.
(23) Kloppenburg, L.; Jones, D.; Bunz, U. H. F. Macromolecules 1999, 32, 4194-4203.
(24) Brizius, G.; Pschirer, N. G.; Steffen, W.; Stitzer, K.; zur Loye, H.-C.; Bunz, U. H. F. J. Am. Chem. Soc. 2000, 122, 12435-12440.
(25) Pschirer, N. G.; Fu, W.; Adams, R. D.; Bunz, U. H. F. Chem. Commun. 2000, 87-88.
(26) Fürstner, A.; Radkowski, K., Chem. Commun. 2002, 2182; Lacombe, F.; Radkowski, K.; Seidel, G.; Fürstner, A. Tetrahedron 2004, 60, 7315; Fürstner, A.; Seidel, G. Angew. Chem. Int. Ed. 1998, 37, 1734; Fürstner, A.; Mathes, C.; Lehmann, C. W. Chem. Eur. J. 2001, 7, 5299; Fürstner, A.; Davies, P. W. Chem. Commun. 2005, 2307; Bunz, U. H. F.; Kloppenburg, L. Angew. Chem. Int. Ed. 1999, 38, 478.
(27) Fürstner, A.; Guth, O.; Rumbo, A.; Seidel, G. J. Am. Chem. Soc. 1999, 121, 11108.
(28) Grela, K.; Ignatowska, J. Org. Lett. 2002, 4, 3747.
(29) Fürstner, A.; Mathes, C.; Lehmann, C. W. J. Am. Chem. Soc. 1999, 121, 9453; Sashuk, V.; Ignatowska, J.; Grela, K. J. Org. Chem. 2004, 69, 7748.
(30) Brizius, G.; Bunz, U. H. F. Org. Lett. 2002, 4, 2829.
(31) Klyatskaya, S.; Dingenouts, N.; Rosenauer, C.; and Höger, S. J. Am. Chem. Soc. 2006, 128, 3150; Ziegler, A.; Mamdouh, W.; Ver Heyen, A.; Surin, M.; Uji-i, H.; Abdel-Mottaleb, M. M. S.; De Schryver, F. C.; De Feyter, S.; Lazzaroni, R.; Höger, S. Chem. Mater. 2005, 17, 5670; Höger, S. Chem. Eur. J. 2004, 10, 1320; Zhao, D.; Moore, J. S. Chem. Commun. 2003, 807; Yamaguchi, Y.; Yoshida, Z. Chem. Eur. J. 2003, 9, 5430; Crave, C.; Schluter, A. D. Eur. J. Org. Chem. 2002, 3075; Höger, S. J. Polym. Sci., Part A: Polym. Chem. 1999, 37, 2685; Prautzsch, V.; Ibach, S.; Vogtle, F. J. Incl. Phenom. Macrocycl. Chem. 1999, 33, 427.
(32) Zhang, W.; Moore, J. S. J. Am. Chem. Soc. 2004, 126, 12796.
(33) Zhang, W.; Brombosz, S. M.; Mendoza, J. L.; Moore, J. S. J. Org. Chem. 2005, 70, 10198.

1. A heterogeneous alkyne metathesis catalyst prepared by a process comprising covalently bonding a precursor to a support, the precursor having structure formula (I): 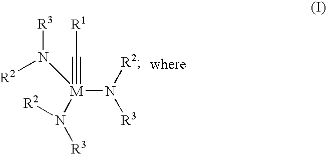
M is a transition metal selected from the group consisting of Mo and W;
R¹ and R² each are independently selected from the group consisting of aliphatic and aromatic moieties; and
R³ is a phenyl group.

2. The catalyst of claim 1, where M is molybdenum.

3. The catalyst of claim 2, where the bonding between the support and the precursor is between an oxygen atom on the support and the transition metal center of the precursor.

4. The catalyst of claim 2, were R¹ and R² each are independently selected from the group consisting of aliphatic and aromatic moieties, which do not contain hydrogen atoms bonded to elements other than carbon.

5. The catalyst of claim 2, where R¹ and R² are independently selected alkyl or cycloalkyl groups.

6. The catalyst of claim 2, where R¹ is ethyl.

7. The catalyst of claim 2, where R² is t-butyl.

8. The catalyst of claim 2, where each R³ is independently substituted with two substituents independently selected from the group consisting of aliphatic groups and aromatic groups.

9. The catalyst of claim 8, were R³ excludes substituents having hydrogen atoms bonded to elements other than carbon.

10. The catalyst of claim 2, where each R³ is independently substituted with two substituents independently selected from the group consisting of aryl, heteroaryl, alkyl, and cycloalkyl.

11. The catalyst of claim 8, where each R³ includes a methyl group at both meta positions.

12. The catalyst of claim 2, where the catalyst is capable of catalyzing alkyne metathesis at temperatures from 15 to 50° C.

13. The catalyst of claim 2, where the catalyst is capable of homodimerizing a 0.28 moles/Liter concentration of a 1-phenyl-1-butyne at 0.3% catalyst loading between 20 and 26° C. with a TOF of at least 0.02 molₚ*mol_{c}⁻¹s⁻¹.

14. The catalyst of claim 2, where the catalyst is capable of homodimerizing a 1-phenyl-1-butyne with a t_{1/2} of less than 20 minutes between 20 and 26° C.

15. A method of metathesizing an alkyne with the catalyst of claim 2, comprising:
reacting the catalyst with the alkyne at a temperature between 15 and 100° C. to metathesize the alkyne.

16. The method of claim 15, where the alkyne includes at least two different alkynes and the two different alkynes undergo homodimerization.

17. The method of claim 15, where the metathesis reaction provides a ring from two alkynes.

18. The method of claim 15, where the metathesis reaction provides a cyclooligomerization product from at least three alkynes.

19. (canceled)

20. A composition having structure formula (III): 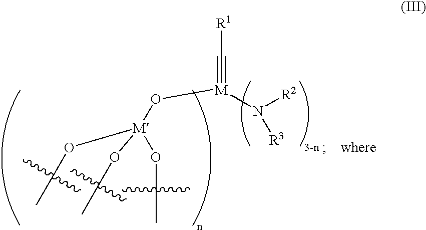
M′ is a metal;
n is an integer from 1 to 3;
M is a transition metal selected from the group consisting of Mo and W;
R¹ and R² each are independently selected from the group consisting of aliphatic and aromatic moieties;
R³ is a phenyl group;
R¹ and R² exclude moieties having functionality selected from the group consisting of hydroxy, primary amine, secondary amine, thiol, sulfoxide, sulfate, phosphine, phosphite, phosphonate, primary silane, secondary silane, tertiary silane, protic acid, and combinations thereof;
R¹ excludes moieties including hydride functionality at the alpha, beta, and gamma positions;
R² excludes moieties having hydride functionality at the alpha and beta positions; and
R³ excludes substituents having functionality selected from the group consisting of hydroxy, primary amine, secondary amine, thiol, sulfoxide, sulfate, phosphine, phosphite, phosphonate, primary silane, secondary silane, tertiary silane, protic acid, and combinations thereof.

21.-31. (canceled)

32. In an alkyne metathesis catalyst having structure formula (I): 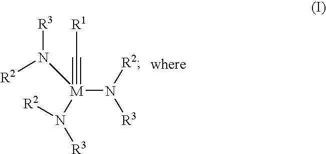
M is a transition metal selected from the group consisting of Mo and W;
R¹ and R² each are independently selected from the group consisting of aliphatic and aromatic moieties; and
R³ is a phenyl group;
the improvement comprising covalently bonding the transition metal center of the catalyst to at least one oxygen atom of a support.

33.-49. (canceled)
